(19) United States
(12) Patent Application Publication
(10) Pub. No.: US 2010/0113974 A1
(43) Pub. Date: May 6, 2010

(54) Method and apparatus for collection of biological samples

(76) Inventor: Larry Williams, Lincoln, NE (US)

Correspondence Address: SUITER SWANTZ PC LLO, 14301 FNB PARKWAY, SUITE 220, OMAHA, NE, 68154, US

(21) Appl. No.: 12/290,915

(22) Filed: Nov. 5, 2008

(51) Int. Cl. A61B 10/00

(52) U.S. Cl. 600/570

(57) ABSTRACT

The present invention provides a device for collection of biological material from the preputial region of a male animal. The basic principles for collection of the biological material are using a collection tip configured for filling the preputial space and scraping the mucosal walls when the device is pulled from the fornix of the prepuce. In one embodiment, the device includes a collection tip, configured for scrapping the mucosal lining of the prepuce, operably connected to an applicator rod.

### FIELD OF THE INVENTION

The present invention is directed generally to collection of biological samples and more particularly, collection of biological samples from the male reproductive tracts.

### BACKGROUND OF THE INVENTION

Central to the diagnosis of diseases of the reproductive organs in cattle is collection of the inciting organism. In the diagnosis of Trichomoniasis and Vibriosis in cattle there are generally no outward signs of the disease until producers begin seeing higher than normal abortion rates. The diagnosis of Trichomoniasis and Vibriosis is best accomplished by a testing program which requires collection of biologic material from the male reproductive tract. Currently, the diagnosis of Trichomoniasis and Vibriosis in the male requires multiple collections, even in infected animals, due to the difficulty in retrieving the inciting organisms. The need to repeatedly collect these animals is both expensive and labor intensive.

Thus, there is a need to provide a more accurate means of collecting biologic samples from the male reproductive tract.

### SUMMARY OF THE INVENTION

Accordingly, an embodiment of the present invention is directed to a biological sample collection device, said device including a collection tip configured for scraping a mucosal surface and collecting a biological sample while being retrieved from the fornix of the prepuce; an applicator, wherein the applicator is a rod operably coupled to the collection tip and configured for directing the collection tip to the fornix of the prepuce through a pushing action and to retrieve same by a opposite pulling action.

It is to be understood that both the foregoing general description and the following detailed description are exemplary only and are not necessarily restrictive of the invention as claimed. The accompanying drawings, which are incorporated in and constitute a part of the specification, illustrate an embodiment of the invention and together with the general description, serve to explain the principles of the invention.

### BRIEF DESCRIPTION OF THE DRAWINGS

The numerous advantages of the present invention may be better understood by those skilled in the art by reference to the accompanying figures in which:

FIG. 1 is an enlarged view of a biological sample device in accordance with an embodiment of the present invention:

FIG. 2 is an enlarged view of a biological sample device, including a protective sleeve, a speculum, and an applicator guide in accordance with an embodiment of the present invention;

FIG. 3 is an enlarged view of a biological sample device, including a protective sleeve, a speculum, and an applicator guide in accordance with an embodiment of the present invention;

FIG. 4 is a sectioned view of a male animal penis and prepuce, including an inserted biological sample device in accordance with an embodiment of the present invention;

FIG. 5A is an enlarged view of a biological sample device with cupping fins in accordance with an embodiment of the present invention;

FIG. 5B is a end-on view of a biological sample device with cupping fins in accordance with an embodiment of the present invention; and

FIG. 6 is a flowchart illustrating a method of for using a biological sample device in accordance with an embodiment of the present invention.

### DETAILED DESCRIPTION OF THE INVENTION

Reference will now be made in detail to the presently preferred embodiments of the invention, examples of which are illustrated in the accompanying drawings.

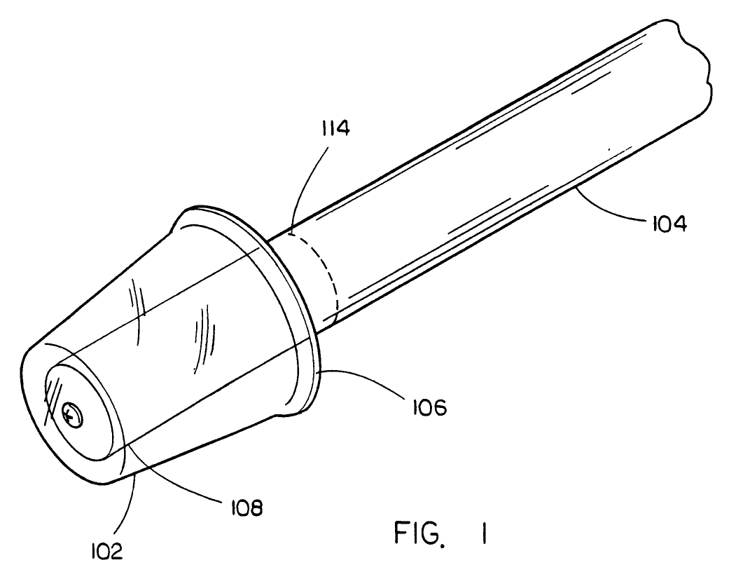

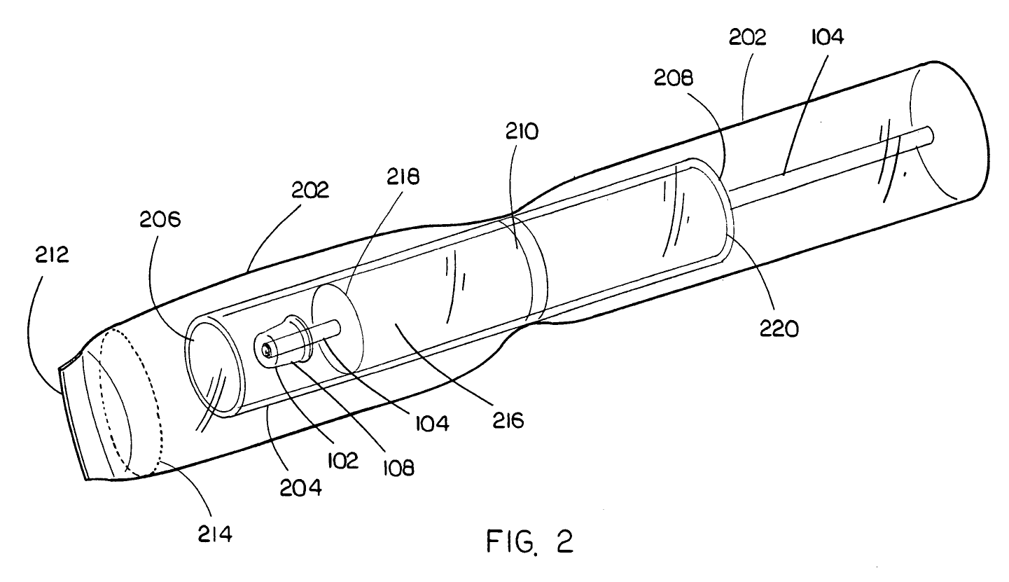

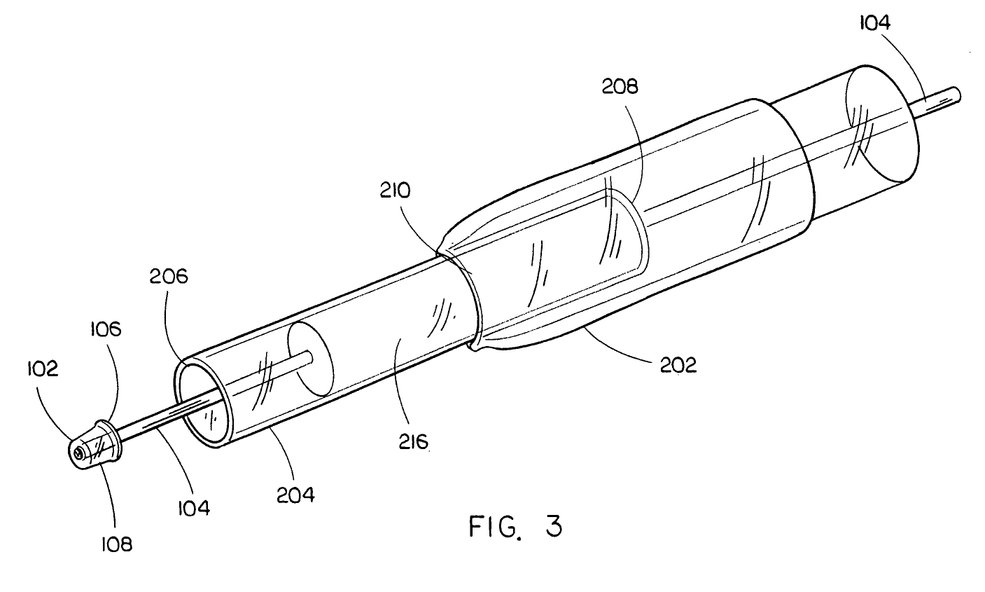

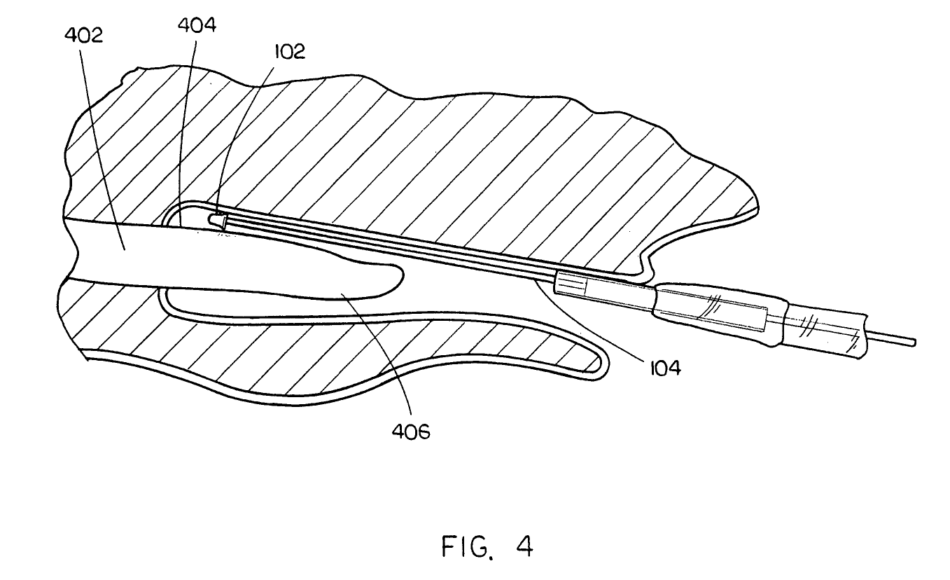

Referring generally to FIGS. 1-4, a biological sample collection device in accordance with an exemplary embodiment of the present invention is shown. The device 100 may include a collection tip 102. The collection tip 102 may be a cup and a vial. The collection tip 102 is configured to collect a biological sample. The collection tip 102 is configured for scraping the mucosal wall 404 of the penis 404 and the mucosal wall of the prepuce 408 when removed from the collection point. For example, to aid in the collection of a biological sample the collection tip rim 106 may be flared with a beveled edge or alternatively the collection tip 102 may include cupping fins 502, or side cut outs/openings of the collection tip. The collection tip 102 of the present invention is conical in shape in order to assist in the ease of placement when inserting the device 100 into the prepuce. The device 100 further includes an applicator 104. The applicator 104 is a rod with a length of between 18 and 21 inches. Alternatively, the applicator 104 may be a pipette.

The applicator 104 is operably coupled 108 to the collection tip 102. For example, the applicator 104 may be operably coupled 108 to the collection tip 102 by a collar connection as shown in FIG. 1. However, the applicator 104 may operably coupled 108 to the collection tip 102 via a suction connection, a rivet connection, a threaded connection, a snap connection and a molded or welded connection. Additionally, the collection tip 102 may be removed from the applicator 104 in order to facilitate the placement of the collection tip 102 into a suitabte media container. The removal of the collection tip 102 from the applicator 104 may be via a reversal of any of the operable coupled 108 techniques including but not limited to breaking of a score line 114 on the applicator 104.

In a further embodiment of the present invention the device 100 may include a speculum 204. The speculum 204 is a tubular structure configured for providing luminal access. The speculum 204 has a first end 206 and a second end 208. The first end 206 is configured with a rounded edge to aid with insertion. When device 100 is used with the speculum 204 the speculum 204 is placed into the opening of the prepuce and the device 100 is advanced into the prepuce without the added risk of contamination from the external edges of the prepuce. The device 100 is advanced through the second end 208 and enters the prepuce at the first end 206. The device is advanced to the fornix 404 of the prepuce.

In a further embodiment of the present invention, the device 100 may include a protective sleeve 202. The protective sleeve 202 may be made of paper or plastic. The protective sleeve 202 is configured for aseptically containing the device 100. For example, the protective sleeve 202 may be a package configured for holding the device 100 including the collection tip 102, the application 104, the speculum 204, and the applicator guide 216. The protective sleeve 202 may further be configured for sterilizing. For example, the protective sleeve 202 may be suitable for gas, radiation, or heat sterilization. Further, the protective sleeve 202 is configured for opening and aseptically presenting the device 100 for use. For example, the protective sleeve 202 may include a tear strip 214, a pressure zipper, an adhesive seal, and a folded end cap.

In a further embodiment of the present invention, the device may include an applicator guide 216. The applicator guide 216 is a tubular structure with a first end 218 and a second end 220. The applicator guide 216 interior is sized to support linear displacement of the applicator 104 when the applicator 104 is advanced through the applicator guide 216. The outer diameter is sized to sealingly fit within the speculum 204 interior. Further, the applicator guide 216 is sealingly retained between the first end 206 and the second end 208 of the speculum. Further, the first end 218 of the applicator guide 216 is recessed from the first end of the speculum 204.

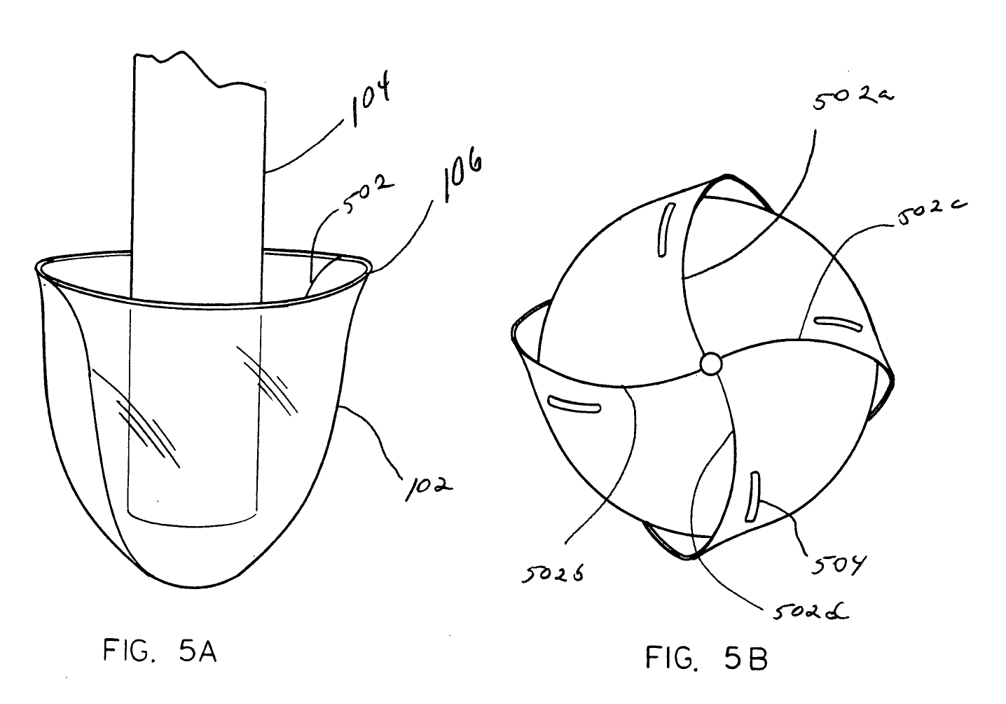

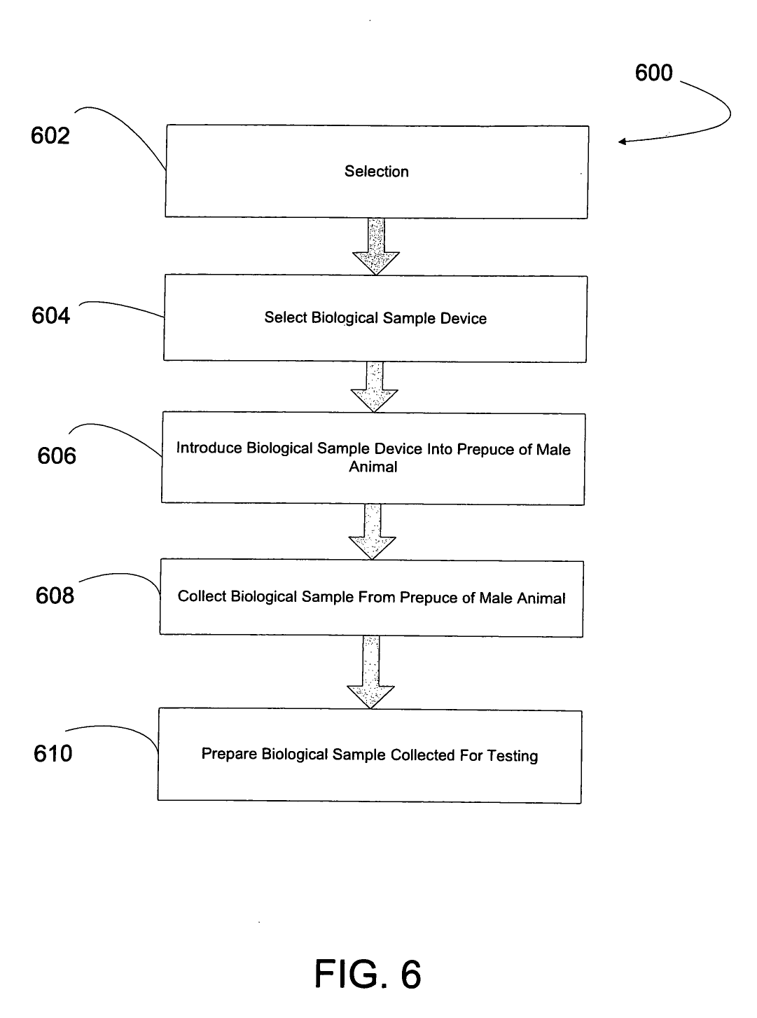

Referring generally to FIG. 6, a method for collecting biological samples is illustrated in accordance with an embodiment of the present invention. The method 600 includes the step of selecting a male animal 602. In a further embodiment of the present invention the step of selecting a biological sample device 604. The step of selecting a biological sample device 604 is generally dependent on the breed, age, and size of the male animal. For example, an older bull with have deeper mucosal folds thereby requiring the use of a device 100 of a larger collection tips 102 size. Further, the method 600 includes the step of introducing the biological sample collection device 606 into the prepuce of a male animal. The step 606 requires restraining the male animal for the procedure and to clean the area of the prepuce where entry will be made. The step 606 then requires the stabilization of the prepuce by placing a hand externally on the wall of the prepuce and inserting the speculum 204 into the prepuce. Step 606 then includes inserting the collection tip 102 through the speculum 204 in a manner as to exit the first end 206 of the speculum 204 and enter into the prepuce; by pushing towards the animal with the applicator 104, advancing the collection tip 102 to the full length of the fornix 404 located at the full depth of the prepuce. Further, the method 600 includes the step of collecting a biological sample from the prepuce of the male animal 608. The step of collecting a biological sample from the prepuce of a male animal 608 includes a pulling on the applicator 104 whereby the collection tip 102 retreats from the fornix 404 of the male animal. The retreat engages the collection tip rim 106 with the mucosal walls 406 of the prepuce and the penis 402 of the male animal. The engagement permits the collection tip rim 106 to reach deep into the mucosal folds and to scrape the smegma material from the mucosa. Alternatively, the collection tip 102 may include cupping fins 502 as illustrated in FIGS. 5A and 5B to aid in collection of smegma material from the recess of the fornix 404.

Further, the method 600 includes the step of preparing the biological sample for testing 610. The step 610 may include removing the sample from the collection tip 102 and place on a microscope or into a container with media suitable for transferring the specific biologic sample. For example, the present invention may provide for the removal of the applicator 104 when the entire collection tip 102 is inserter into a media filled vial suitable for seating and transporting.

It is understood that the specific order or hierarchy of steps in the foregoing disclosed methods are examples of exemplary approaches. Based upon design preferences, it is understood that the specific order or hierarchy of steps in the method can be rearranged while remaining within the scope of the present invention. The accompanying method claims present elements of the various steps in a sample order, and are not meant to be limited to the specific order or hierarchy presented.

It is further understood that the collection of biological material is not limited to the male species. The accompanying drawings are for illustration of the biological collection device 100 with the male species referenced for clarity, however collection of biological material from the female of the species may be accomplished with use of the biological collection device 100.

It is believed that the present invention and many of its attendant advantages are to be understood by the foregoing description, and it is apparent that various changes may be made in the form, construction and arrangement of the components thereof without departing from the scope and spirit of the invention or without sacrificing all of its material advantages. The form herein before described being merely an explanatory embodiment thereof, it is the intention of the following claims to encompass and include such changes.

What is claimed is:

1. A biological sample collection device, comprising:
a collection tip, wherein the collection tip is configured for scraping a mucosal surface and collecting a biological sample while being pulled from a collection location; and
an applicator, wherein the applicator is a rod operably connected to the collection tip.

2. The device of claim 1 wherein the collection tip is at least one of a cup, a vial, and a tube.

3. The device of claim 1 wherein the collection tip is conical in shape with an open end.

4. The device of claim 1 wherein the collection tip has a flared and beveled edge at a collection point.

5. The device of claim 1 wherein the collection tip is configured for collecting a biological sample via scrapping a mucosal surface of a animal.

6. The device of claim 1 wherein the applicator is operably connected to the collection tip via at least one of a suction connection, a rivet connection, a threaded connection, a snap connection, a molded connection, and a welded connection.

7. The device of claim 1 further comprising:
a speculum, wherein the speculum is a tubular structure configured for providing luminal access.

8. The device of claim 1 further comprising:
a plastic sleeve, wherein the plastic sleeve is configured for aseptically containing at least one of the collection tip, the applicator, and a speculum.

9. A biological sample collection device, comprising:
a collection tip, wherein the collection tip is configured for scraping a mucosal surface and collecting a biological sample while being pulled from a collection location;
an applicator, wherein the applicator is a rod operably connected to the collection tip;
a protective sleeve, wherein the protective sleeve is configured for containing the biological sample collection device in an aseptic environment; and
a speculum, wherein the speculum is a tubular structure including a first end and a second end and configured for avoidance of surface debris while providing the biological sample collection device access to luminal areas of the body.

10. The biological sample collection device of claim 9, wherein the collection tip is a container with a receiving edge that is flanged and beveled for scraping a mucosal surface.

11. The biological sample collection device of claim 9, wherein the applicator is operably connected to the collection tip via at least one of a suction connection, a rivet connection, a threaded connection, a snap connection, a welded connection, and a molded connection.

12. The biological sample collection device of claim 9, wherein the protective sleeve is coupled to the speculum.

13. The biological sample collection device of claim 9, wherein the protective sleeve is seated with a means for opening and aseptically presenting the biological sample collection device.

14. The biological sample collection device of claim 9, wherein the first end of the speculum is rounded to aid insertion of the speculum.

15. The biological sample collection device of claim 9, further comprising:
a applicator guide including a first end and a second end, wherein the applicator guide interior diameter is sized to support Linear displacement of the applicator and the outer diameter is sized to sealingly fit within the speculum orifice;
the applicator guide sealingly retained between the first end of the speculum and the second end of the speculum, wherein the first end of the applicator guide is recessed from the first end of the speculum.

16. A method for collecting biological samples, comprising:
selecting a male animal;
introducing the biological sample device into the prepuce of the male animal;
collecting a biological sample from the prepuce of the male animal by withdrawal of the biological sample device from the male animal; and
preparing the biological sample from the prepuce of the male animal for testing.

17. The method as claimed in claim 16, wherein introducing the biological sample device into the prepuce of the male animal comprises:
placing a hand externally on the wall of the prepuce to control the placement of the biological sample device;
inserting a speculum into the opening of the prepuce of the male animal;
passing the biological sample device through the speculum and into the prepuce; and
advancing the biological sample device to the full length of the preputial cavity.

18. The method as claimed in claim 16, wherein introducing the biological sample device into the prepuce of the male animal comprises:
placing a hand externally on the wall of the prepuce to control the placement of the biological sample device;
inserting the biological sample device through the opening of the prepuce of the male animal;
advancing the biological sample device to the full length of the preputial cavity.

19. The method as claimed in claim 16, wherein collecting a biological sample from the prepuce of the male animal by withdrawal of the biological sample device from the male animal comprises:
scraping the surface of the penis and the surrounding preputial tissues by a forward and back movement of the biological sample device; and
incremental withdrawal of the biological sample device from the prepuce of the male animal.

20. The method as claimed in claim 16, wherein preparing the biological sample from the prepuce of the male animal for testing comprises:
removing a biological sample from the biological sample device;
opening transport container;
placing biological sample in transport container; and
closing transport container.
